(19) United States
(12) Patent Application Publication
(10) Pub. No.: US 2023/0233761 A1
(43) Pub. Date: Jul. 27, 2023

(54) SYSTEMS AND METHODS FOR PREVENTING BACKFLOW IN A CATHETER SYSTEM

(71) Applicant: Becton, Dickinson and Company, Franklin Lakes, NJ (US)

(72) Inventors: Gokulrangarajan Sathyanarayanan, Coimbatore (IN); Harikrishnan S., Madurai (IN)

(21) Appl. No.: 18/075,211

(22) Filed: Dec. 5, 2022

Related U.S. Application Data
(60) Provisional application No. 63/302,448, filed on Jan. 24, 2022.

(51) Int. Cl. A61M 5/168; A61M 25/00

(52) U.S. Cl. CPC A61M 5/16881; A61M 25/0097; A61M 2005/1406

(57) ABSTRACT

A catheter assembly may include a catheter, a catheter hub coupled to the catheter, an injection port on the catheter hub, and an injection port backflow control device. The backflow control device may include a concentric port, a bellow, and a duckbill valve. The injection port backflow control device may include a split septum and a duckbill valve. The injection port backflow control device may include a deformable annular valve disposed around an inner surface of the catheter hub and may block an opening in the catheter hub connected to the injection port. The injection port may receive a needleless syringe for administration of medicine or other fluids. The injection port backflow control device may permit fluids to flow into the catheter hub and may prevent fluids from flowing out of the catheter hub through the injection port.

### RELATED APPLICATIONS

This application claims the benefit of U.S. Provisional Pat. Application No. 63/302,448, filed Jan. 24, 2022, and entitled SYSTEMS AND METHODS FOR PREVENTING BACKFLOW IN A CATHETER SYSTEM, which is incorporated herein in its entirety.

### BACKGROUND

A common type of catheter assembly includes a peripheral intravenous catheter (“PIVC”) that is over-the-needle. As its name implies, the PIVC that is over-the-needle may be mounted over an introducer needle having a sharp distal tip. The catheter assembly may include a catheter hub, the PIVC extending distally from the catheter hub, and the introducer needle extending through the PIVC. The PIVC and the introducer needle may be assembled such that the distal tip of the introducer needle extends beyond the distal tip of the PIVC with the bevel of the needle facing up away from skin of the patient immediately prior to insertion into the skin. The PIVC and the introducer needle are generally inserted at a shallow angle through the skin into vasculature of the patient.

In order to verify proper placement of the introducer needle and/or the PIVC in the blood vessel, a clinician may confirm that there is flashback of blood in a flashback chamber of the catheter assembly. Once placement of the introducer needle has been confirmed, the clinician may remove the introducer needle, leaving the PIVC in place for future blood withdrawal or fluid infusion.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices and related methods. More particularly, the present disclosure relates to a catheter assembly and related methods. In some embodiments, the catheter assembly may include a catheter, a catheter hub coupled to the catheter, an injection port disposed on the catheter hub, and an injection port backflow control device. In some embodiments, the catheter assembly may include a peripheral intravenous catheter assembly. In some embodiments, the injection port backflow control device may include a concentric port, a bellow, and a duckbill valve. In some embodiments, the injection port may be configured to receive a needleless syringe for administration of medicine or other fluids. In some embodiments, the injection port backflow control device may permit fluids to flow into the catheter hub and may prevent fluids from flowing out of the catheter hub through the injection port.

In some embodiments, the bellow may block a sidewall opening of the concentric port when the bellow is in an expanded state. In some embodiments, the bellow may be vertically compressible from the expanded state to a compressed state. In some embodiments, the bellow, in the compressed state, may permit fluids to flow into the sidewall opening of the concentric port. In some embodiments, the bellow may surround the concentric port and may cover the sidewall opening in the concentric port when the bellow is in the expanded state or not compressed. In some embodiments, the concentric port may include a vertical opening that may connect the sidewall opening with an opening in the base of the concentric port. In some embodiments, the opening in the base of the concentric port may connect with an opening in the duckbill valve. In some embodiments, the duckbill valve opening may permit a fluid to flow into the catheter hub.

In some embodiments, a fluid pathway within the injection port backflow control device may include the sidewall opening in the concentric port, the annular opening, and the duckbill valve. In some embodiments, the vertical opening may be in the center of the concentric port and may connect the sidewall opening to a top of the duckbill valve. In some embodiments, the duckbill valve may be configured to expand to permit passage of a fluid into the catheter hub.

In some embodiments, a peripheral intravenous catheter assembly may include a catheter, a catheter hub coupled to the catheter, an injection port on the catheter hub, and an injection port backflow control device. In some embodiments, the injection port backflow control device may include a split septum and a duckbill valve. In some embodiments, the split septum may include multiple leaves. In some embodiments, the injection port may receive a needleless syringe. In some embodiments, the injection port backflow control device may permit fluids to flow into the catheter hub and prevent fluids from flowing out of the catheter hub through the injection port.

In some embodiments, the duckbill valve may be positioned within the injection port, and a line formed by a meeting of opposing lips of the duckbill valve may be oriented downward towards the catheter hub. In some embodiments, the split septum may be placed as a cap over a top opening of the injection port, and the leaves of the split septum may be deformable to open downwards towards the catheter hub. In some embodiments, a fluid pathway through the injection port and into the catheter may include an opening in the split septum that may be connected to an opening in the duckbill valve. In some embodiments, the duckbill valve may be configured to expand to allow a fluid to flow into the catheter hub and through the catheter hub into the catheter.

In some embodiments, a peripheral intravenous catheter assembly may include a catheter, a catheter hub coupled to the catheter, an injection port on the catheter hub and an injection port backflow control device. In some embodiments, the injection port backflow control device may include a deformable annular valve disposed around an inner surface of the catheter hub and may block an opening in the catheter hub connected to the injection port.

In some embodiments, the injection port may be configured to receive a needleless syringe. In some embodiments, the injection port backflow control device may permit fluids to flow into the catheter hub and may prevent fluids from flowing out of the catheter hub through the injection port.

In some embodiments, the annular valve may deform longitudinally from a fluid pressure of a fluid injected through the injection port into the catheter hub. In some embodiments, the annular valve may include a projection from an outer sidewall of the annular valve. In some embodiments, the catheter hub may include a notch that corresponds to the projection on the annular valve. In some embodiments, the notch and the projection may be engaged to secure the annular valve within the catheter hub.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is a side view of an example peripheral intravenous catheter assembly, according to some embodiments;

FIG. 2 is an upper perspective view of the peripheral intravenous catheter assembly of FIG. 1 after insertion into an arm of a patient, according to some embodiments;

FIG. 3 is an upper perspective view of the peripheral intravenous catheter assembly of FIG. 1 after insertion into the arm of the patient showing injection of a fluid into the peripheral intravenous catheter assembly through an example injection port, according to some embodiments;

FIG. 4 is an upper perspective view of a proximal end of an example peripheral intravenous catheter assembly with an example injection port backflow control device, according to some embodiments;

FIG. 5 is a cross-sectional view of the injection port backflow control device in the peripheral intravenous catheter assembly of FIG. 4;

FIG. 6 is an upper perspective view of an example concentric port, according to some embodiments;

FIG. 7 is a lower perspective view of an example bellow, according to some embodiments;

FIG. 8 is a side view of an example duckbill valve, according to some embodiments;

FIG. 9A is a cross-sectional view of the injection port backflow control device in the peripheral intravenous catheter system of FIG. 4, according to some embodiments;

FIG. 9B is a cross-sectional view of the injection port backflow control device in the peripheral intravenous catheter system of FIG. 4, illustrating the flow of fluids from a needleless syringe into the peripheral intravenous catheter system through an example injection port, according to some embodiments;

FIG. 10 is an upper perspective view of an example peripheral intravenous catheter assembly with an example injection port backflow control device, according to some embodiments;

FIG. 11 is a cross-sectional view of the peripheral intravenous catheter assembly of FIG. 10;

FIG. 12 is a lower perspective view of an example split septum, according to some embodiments;

FIG. 13 is an upper perspective view of an example duckbill valve, according to some embodiments;

FIG. 14 is a cross-sectional view of the peripheral intravenous catheter assembly of FIG. 10, illustrating a needleless syringe inserted into the injection port, according to some embodiments;

FIG. 15 is a cross-sectional view of an example injection port backflow control device in an example peripheral intravenous catheter assembly, illustrating flow of fluids into the peripheral intravenous catheter assembly through an example port, according to some embodiments;

FIG. 16 is a cross-sectional view of an example injection port backflow control device in an example peripheral intravenous catheter assembly, according to some embodiments;

FIG. 17A is an upper perspective view of an example valve, according to some embodiments;

FIG. 17B is a side view of the valve of FIG. 17A, according to some embodiments;

FIG. 18 is an isometric cross-sectional view of an example injection port backflow control device in an example peripheral intravenous catheter assembly, according to some embodiments;

FIG. 19A is a left-side upper perspective view of an example valve, according to some embodiments;

FIG. 19B is a side view of the valve of FIG. 19A, according to some embodiments; and

FIG. 19C is a right-side upper perspective view of the valve of FIG. 19A, according to some embodiments.

### DESCRIPTION OF EMBODIMENTS

The present disclosure relates generally to vascular access devices and related methods. More particularly, the present disclosure relates to a catheter assembly and related methods. In some embodiments, the catheter assembly, may include a catheter, a catheter hub coupled to the catheter, an injection port on the catheter hub, and an injection port backflow control device. In some embodiments, the backflow control device may include a concentric port, a bellow, and a duckbill valve. In some embodiments, the injection port may receive a needleless syringe for administration of medicine or other fluids. In some embodiments, the injection port backflow control device may permit fluids to flow into the catheter hub and may prevent fluids from flowing out of the catheter hub through the injection port.

Referring now to FIGS. 1-3, in some embodiments, a peripheral intravenous catheter assembly 100 may be configured to deliver fluids to a patient through a peripheral blood vessel. In some embodiments, the peripheral intravenous catheter assembly 100 may include an injection port for injection of fluids into the patient. In some embodiments, the peripheral intravenous catheter assembly 100 may have a catheter 130 on a distal end for insertion into a patient. In some embodiments, the catheter 130 may include a peripheral intravenous catheter. In some embodiments, the catheter 130 may be connected to a catheter hub 110. In some embodiments, the catheter hub 110 may have a lumen for flow of blood and other fluids into or out of the patient. In some embodiments, the proximal end of the catheter hub 110 may have an opening through which a needle may be inserted for facilitating insertion of the catheter 130 into the patient. In some embodiments, the needle may be removed after insertion and an end cap 105 may be attached to the proximal end of the catheter hub 110, closing the proximal end of the catheter hub 110.

According to some embodiments, the peripheral intravenous catheter assembly 100 may include the injection port disposed on the top of the catheter hub 110. In some embodiments, the port may be covered with an injection port cap 115. In some embodiments, a valve 120 may be positioned in the lumen of the catheter hub 110. In some embodiments, the valve 120 may prevent fluids from flowing out of the injection port while permitting fluids to flow through the injection port and into the catheter hub 110 and the catheter 130. In some embodiments, the peripheral intravenous catheter assembly 100 may include wings 125 to stabilize the assembly against the skin of a patient.

In some embodiments, in response to the catheter being placed in a vein, the clinician may observe flashback in the catheter 130 and/or another portion of the peripheral intravenous catheter assembly 100. Flashback may include blood flowing into the catheter 130 from the patient. In some embodiments, the force of the patient’s blood flowing into the catheter 130 and then into the catheter hub 110 may push the valve 120 in the proximal direction. In some embodiments, after the valve 120 has been proximally displaced, the valve 120 may no longer seal the injection port 145 and the patient’s blood may flow up through the injection port 145 and out of the peripheral intravenous catheter assembly 100. In some embodiments, other fluids flowing through the peripheral intravenous catheter assembly 100 or other forces may cause the valve 120 to be displaced within the peripheral intravenous catheter assembly 100 and cause the valve 120 to malfunction.

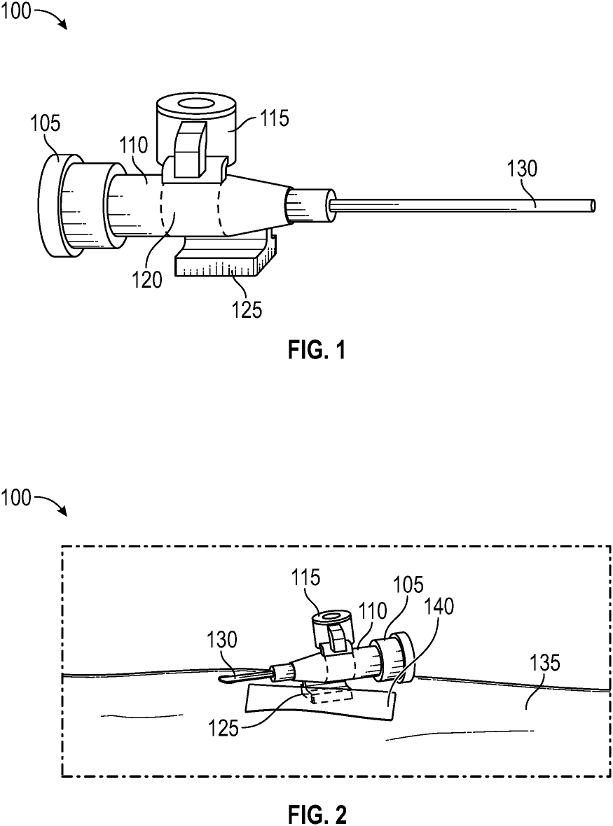

In FIG. 2, the peripheral intravenous catheter assembly 100 is illustrated in use, after insertion into an arm of a patient, according to some embodiments. In some embodiments, the catheter 130 may be inserted into the arm of the patient 135. In some embodiments, the wings 125 may be folded downwards and secured to the patient’s arm 135 by an adhesive tape 140. In some embodiments, the catheter 130 may provide access to a patient’s vasculature for administration of fluids, medications, and blood transfusions. In some embodiments, fluids, medications, and a blood transfusion may be injected into the peripheral intravenous catheter assembly 100 through the injection port 145.

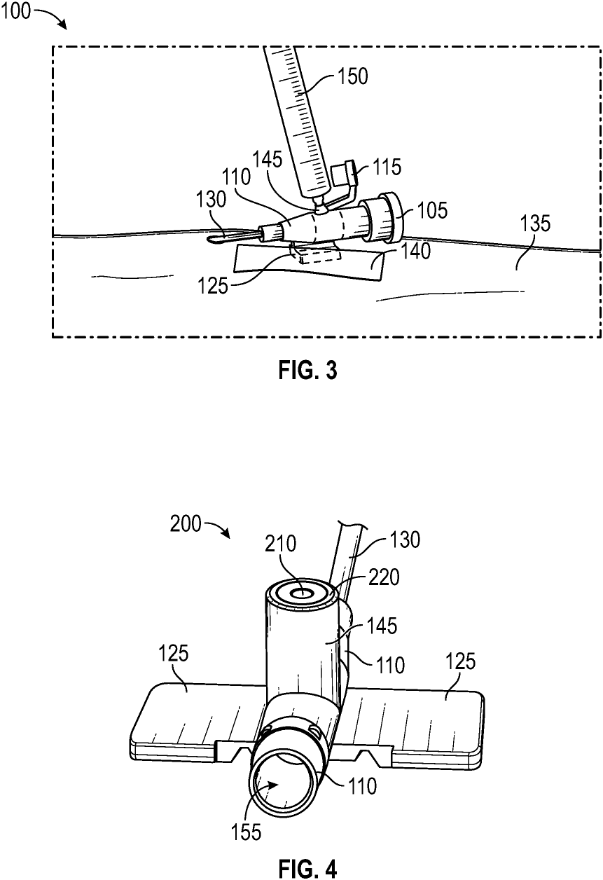

In FIG. 3, the peripheral intravenous catheter assembly 100 is illustrated after insertion into the arm of the patient, according to some embodiments. In some embodiments, fluids may be injected into the peripheral intravenous catheter assembly 100 through the injection port 145, according to some embodiments. In some embodiments, a needleless syringe 150 or another suitable infusion device may be used to inject fluids into the injection port 145.

Referring now to FIGS. 4-9, a peripheral intravenous catheter assembly 200 with an injection port backflow control device is illustrated, according to some embodiments. In some embodiments, the peripheral intravenous catheter assembly 200 may be similar or identical to the peripheral intravenous catheter assembly 100 in terms of one or more components and/or operation. As illustrated, for example, in FIG. 4, in some embodiments, the peripheral intravenous catheter assembly 200 may include the catheter hub 110 with a lumen 155 disposed within the catheter hub 110. In some embodiments, the wings 125 may extend longitudinally from the catheter hub 110. In some embodiments, the injection port 145 may extend vertically from the top of the catheter hub 110. In some embodiments, the injection port 145 may be used to inject fluids into the catheter hub 110 and then into the catheter 130 for infusion into a patient’s body. In some embodiments, the injection port 145 may be used for administration of intravenous fluids, various medicines, or for blood transfusions.

An injection port backflow control device is illustrated installed within the injection port 145, according to some embodiments. In some embodiments, the injection port backflow control device may include multiple pieces. According to some embodiments, the injection port 145 may be sealed when the needleless syringe is removed from the injection port 145.

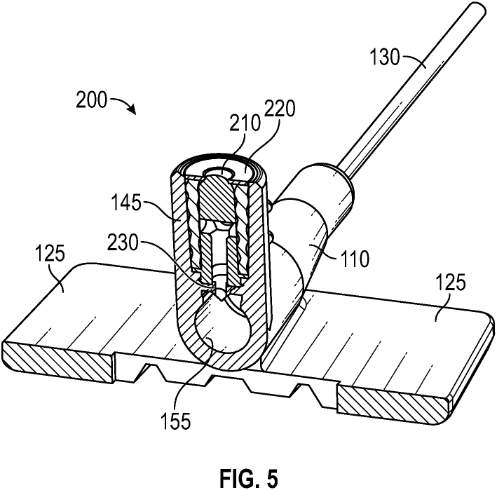

In FIG. 5, a cross-sectional view of the injection port backflow control device within the peripheral intravenous catheter assembly 200, is illustrated, according to some embodiments. In some embodiments, the injection port backflow control device may be positioned within the injection port 145. In some embodiments, the injection port backflow control device may include three pieces. In some embodiments, the injection port backflow control device may include a concentric port 210, a bellow 220, and a duckbill valve 230. In some embodiments, the concentric port 210 may be positioned within the bellow 220 such that the bellow surrounds the concentric port 210. In some embodiments, the duckbill valve 230 may be positioned at the base of the concentric port 210. In some embodiments, opposing lips of the duckbill valve 230 may open to allow a fluid to flow from the opening in the concentric port 210 through an opening in the duckbill valve 230. In some embodiments, the duckbill valve 230 may point downwards such that when the opposing lips are opened, the opening in the duckbill valve 230 may be fluidically connected to a lumen or opening in the concentric port 210, which may be in a center of the concentric port 210. In some embodiments, the lumen or opening of the concentric port 210 may include a vertical opening. In some embodiments, the fluid pressure may cause the opposing lips of the duckbill valve 230 to open and may permit the fluid to flow into the lumen 155 of the catheter hub 110.

In some embodiments, the injection port backflow control device may include at least one of: the concentric port 210, the bellow 220, and the duckbill valve 230. In some embodiments, the injection port backflow control device may include the concentric port 210 and the bellow 220. In some embodiments, the injection port backflow control device may include the concentric port 210 and the duckbill valve 230. In some embodiments, the injection port backflow control device may include the bellow 220 and the duckbill valve 230. In some embodiments, the injection port backflow control device may include a concentric port 210. In some embodiments, the injection port backflow control device may include the bellow 220. In some embodiments, the injection port backflow control device may include the duckbill valve 230. In some embodiments, the injection port backflow control device may include other parts in addition to the concentric port 210, the bellows 220, and the duckbill valve 230.

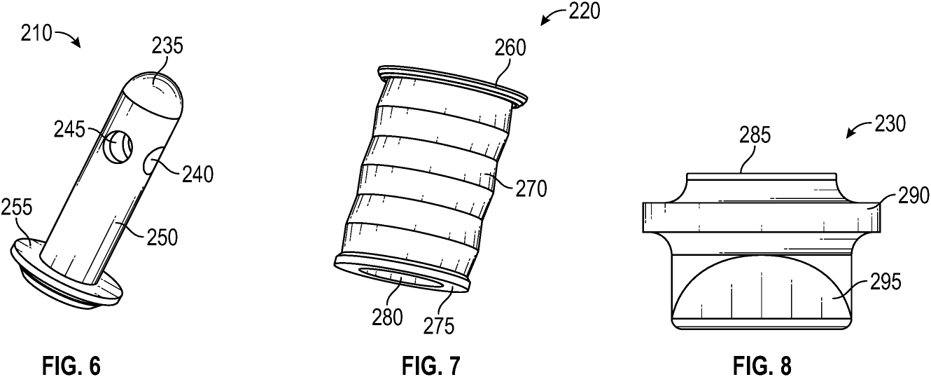

In FIG. 6, the concentric port 210 is illustrated, according to some embodiments. In some embodiments, the concentric port 210 may have one or more sidewall openings in a body 250 of the concentric port 210. In some embodiments, the sidewall openings may permit the fluid to flow into the lumen the concentric port 210. In some embodiments, the fluid may then flow down through the lumen, which may be in the center of the concentric port 210, and exit the concentric port 210 at an opening in the base 255 of the concentric port 210. In some embodiments, the sidewall openings may include first sidewall opening 240 and/or a second sidewall opening 245, which may facilitate fluid flow through the concentric port 210. In some embodiments, the base 255 of the concentric port 210 may be surrounded by a ring, the ring providing a stable platform for the concentric port 210. In some embodiments, the bellow 220 may rest on top of the ring and the duckbill valve 230 engage with the bottom of the ring.

In FIG. 7, in some embodiments, the bellow 220 may include a compressible body 270 that is cylindrical in shape. In some embodiments, the compressible body 270 may include an accordion shape with multiple annular folds. In some embodiments, the bellow 220 may include an upper lip 260 and a lower lip 275. In some embodiments, the bellow 220 may be vertically compressible. In some embodiments, the bellow 220 may surround the outer surface of the concentric port 210. In some embodiments, the bellow 220 may be positioned between the outer surface of the concentric port 210 and an inner surface of the injection port 145.

According to some embodiments, the needleless syringe may be inserted into the injection port 145. In some embodiments, the needleless syringe may contact the upper lip 260 of the bellow 220. In some embodiments, the bellow 220 may be depressed in a vertical direction and towards the lumen 155 using the needleless syringe 150. In some embodiments, the bellow 220 may be compressed by the needleless syringe, thus exposing the first sidewall opening 240 and the second sidewall port 245. In some embodiments, the needleless syringe may be in fluid contact with the first and second sidewall openings 240 and 245 of the concentric port 210. In some embodiments, a fluid pathway may be defined or created by compressing the bellow 220, the fluid flowing through the first sidewall opening 240 and the second sidewall opening 245 into the lumen in the center of the concentric port 210. In some embodiments, the fluid from the needleless syringe may open the duckbill valve 230 and flow into the lumen 155 of the catheter hub 110.

In FIG. 8, a side view of the duckbill valve 230, is illustrated, according to some embodiments. In some embodiments, the duckbill valve 230 may include an opening 285 in the top of the duckbill valve 230. In some embodiments, the opening 285 may be configured to permit the fluid to flow through the center of the duckbill valve 230. In some embodiments, the duckbill valve 230 may include the opposing lips 295. In some embodiments, the opposing lips 295 may open to allow fluid to flow through the duckbill valve 230 from the top to the bottom of the duckbill valve 230.

In some embodiments, the opposing lips 295 may facilitate opening of the duckbill valve 230 to allow the fluid into the lumen 155 in response to fluid pressure from the needleless syringe 150 and closing of the duckbill valve 230 to prevent fluid from flowing from the lumen 155 through the injection port 145. In some embodiments, the opposing lips 295 may remain in a closed position until a fluid pressure forces them open when the fluid flows down through the center of the duckbill valve 230. In some embodiments, a fluid flowing in the reverse direction from the catheter hub 110 into the injection port 145 would be stopped by the opposing lips 295.

In some embodiments, the duckbill valve 230 may further include a ring 290. In some embodiments, the ring 290 may be configured to engage with a base 255 of the concentric port 210. In some embodiments, the ring 290 may be configured to engage with a surface in the injection port 145 such that the ring 290 rests on the surface of the injection port 145. In some embodiments, the injection port 145 may have a congruent ring configured to receive the duckbill valve 230 ring 290 such that the duckbill valve 230 remains seated within the injection port 145 during use.

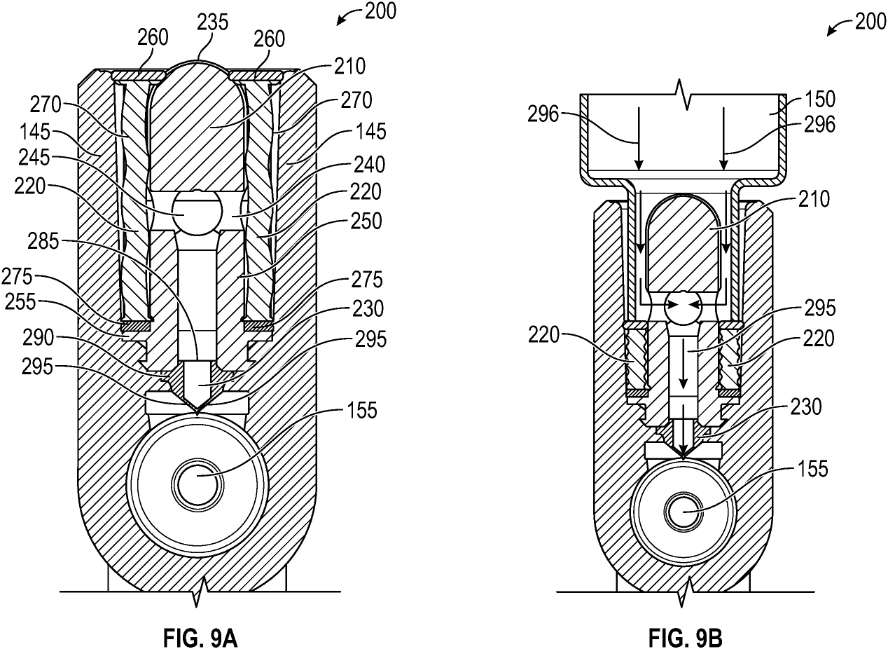

In FIG. 9A, the cross-sectional view illustrates the injection port backflow control device fully assembled within the injection port 145. In some embodiments, the top of the injection port 145 may be open. In some embodiments, the injection port backflow control device may be positioned within an opening of the injection port 145. In some embodiments, the concentric port 210 may be positioned in the center of the injection port 145. In some embodiments, the concentric port may be referred to as “the concentric port” because it may be concentric with one or more of the bellow 220, the injection port 145, and the duckbill valve 230. In some embodiments, the first sidewall opening 240 and second sidewall opening 245 may be positioned longitudinally through the middle of the concentric port 210. In some embodiments, the top 235 of the concentric port 210 may be slightly above, even with, or slightly below the top of the injection port 145. In some embodiments, the base 255, which may be annular, of the concentric port 210 may rest on a protrusion in the injection port 145.

In some embodiments, the bellow 220 may be positioned between the inner surface of the injection port 145 and the outer surface of the concentric port 210. The bellow 220 may be configured to compress vertically. The upper lip 260 of the bellow 220 may be flush with the top surface of the injection port 145. The lower lip 275 of the bellow 220 may rest on the base 255 of the concentric port 210. In some embodiments, when the bellow 220 is compressed, the first sidewall opening 240 and the second sidewall opening 245 may be exposed.

In some embodiments, the duckbill valve 230 may be positioned at the base of the concentric port 210. In some embodiments, the opposing lips 295 of the duckbill valve 230 may open when the fluid is injected through the top of the injection port 145. In some embodiments, the opposing lips 295 of the duckbill valve 230 may be in a default closed position when a fluid flows from the lumen 155 of the catheter hub up through the injection port 145. In some embodiments, the duckbill valve 230 remains in a closed position at all times unless there is fluid pressure from the fluid flowing into the opening of the injection port 145.

In FIG. 9B, a cross-sectional view of the injection port backflow control device in the peripheral intravenous catheter assembly 200, is illustrated, according to some embodiments. In some embodiments, arrows 296 the direction of flow of fluids from a needleless syringe 150 into the peripheral intravenous catheter assembly 200 through an injection port 145 is illustrated, according to some embodiments. In some embodiments, the needleless syringe 150 may compress the bellow 220. In some embodiments, the bellow 220 may completely fill the space between the injection port 145 and the concentric port 210. In some embodiments, the fluid may flow around the top of the concentric port 210 and into the first sidewall opening 240 and the second sidewall opening 245. In some embodiments, the fluid may then flow down a lumen in the concentric port 210 and into the duckbill valve 230. In some embodiments, the fluid pressure may cause the opposing lips 295 of the duckbill valve 230 to open and the fluid to flow out of the duckbill valve 230 and into the lumen 155 of the catheter hub 110.

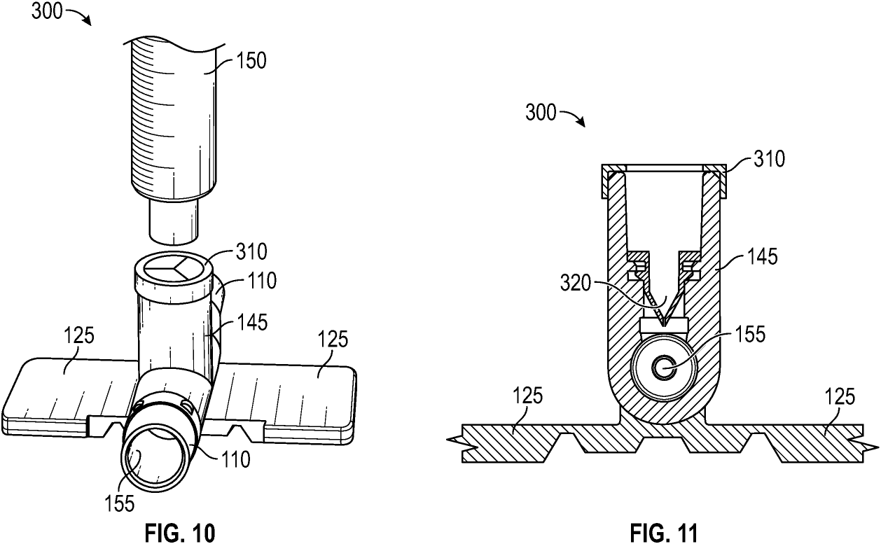

Referring now to FIGS. 10-14, a peripheral intravenous catheter assembly 300 with an injection port backflow control device is illustrated, according to some embodiments. In some embodiments, the peripheral intravenous catheter assembly 300 may be similar or identical to the peripheral intravenous catheter assembly 100 and/or the peripheral intravenous catheter assembly 200 in terms of one or more components and/or operation. In some embodiments, the injection port 145 may be configured to receive the needleless syringe 150. The top of the injection port 145 may be covered by a split septum 310. In some embodiments, the injection port 145 may be connected to a catheter hub 110. In some embodiments, the catheter hub 110 may include the lumen 155. In some embodiments, fluids may flow from the needleless syringe 150 into the injection port 145, then into the lumen 155 of the catheter hub 110 and then out through the catheter 130 and into a patient.

In FIG. 11, the injection port backflow control device in the peripheral intravenous catheter assembly 300 is illustrated, according to some embodiments. According to some embodiments, the injection port backflow control device may include a split septum 310 and a duckbill valve 320. In some embodiments, the split septum 310 may be positioned on the top of the injection port 145. In some embodiments, the duckbill valve 320 may be positioned within the injection port 145. In some embodiments, the duckbill valve 320 may open when fluids flow into the injection port 145 and may remain in a closed position at other times, thus preventing fluids from flowing out of the injection port 145. In some embodiments, the injection port backflow control device may include additional parts in addition to the split septum 310 and the duckbill valve 320. In some embodiments, the injection port backflow control device may include only a split septum 310. In some embodiments, the injection port backflow control device may include only a duckbill valve 320.

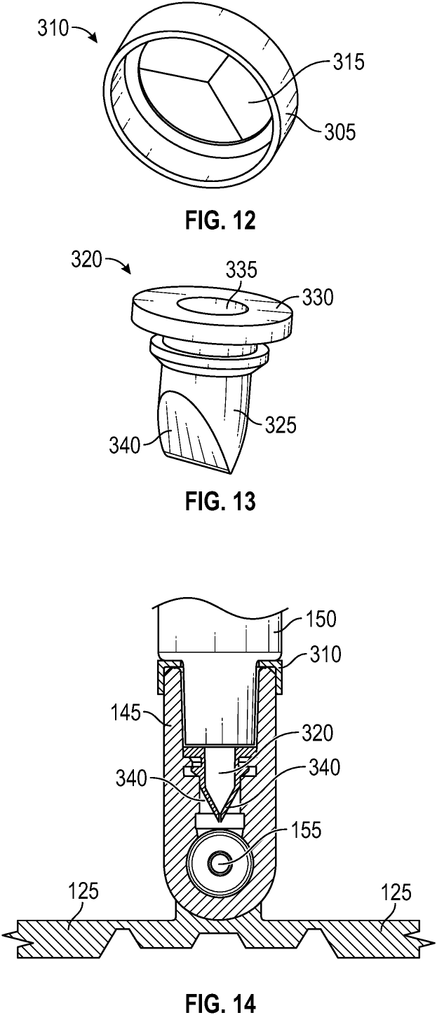

In FIG. 12, the split septum 310 is illustrated, according to some embodiments. In some embodiments, the split septum 310 may include a ring 305 that sits on the upper edge of the injection port 145 and around the outside of the upper edge of the injection port 145. In some embodiments, the ring 305 may have a friction fit on the top of the injection port 145 such that the split septum 310 remains in place on top of the injection port 145 and prevents fluids from leaking out of the sides of the split septum 310 or between the split septum 310 and the injection port 145. In some embodiments, the center of the split septum 310 may include multiple leaves 315 that may be deformable downward. In some embodiments, the leaves 315 may deform downward when a needleless syringe 150 is inserted into the injection port 145. In some embodiments, after the needleless syringe 150 is removed from the injection port 145 the leaves 315 of the split septum 310 may return to their original position and seal off the injection port 145, preventing fluids from seeping out of the injection port 145.

In FIG. 13, the duckbill valve 320 is illustrated, according to some embodiments. In some embodiments, the duckbill valve 320 may include an opening 335 through the top that is connected to a central cavity that passes through the duckbill valve 320 and is closed by opposing lips 340 of the duckbill valve 320. In some embodiments, the top of the duckbill valve 320 may include a ring 330 that may engage with a structure in the injection port 145. In some embodiments, the ring 330 may seat the duckbill valve within the injection port 145 and may maintain the duckbill valve 320 in the injection port 145 when fluids flow through the injection port 145. In some embodiments, the duckbill valve 320 may include an elongated or shortened body 325 based on the length of the injection port 145. In some embodiments, the interior of the duckbill valve 320 may include a central cavity. In some embodiments, in a first state the opposing lips 340 of the duckbill valve 320 may be closed, preventing fluids from flowing out of the injection port 145 from the catheter hub 110. In some embodiments, in a second state, the opposing lips 340 of the duckbill valve 320 may open to permit fluids to flow into the peripheral intravenous catheter assembly 300. In some embodiments, the opposing lips 340 of the duckbill valve 320 remain in a closed position unless acted upon by a fluid flowing into the injection port 145 through the top opening.

In FIG. 14, the injection port backflow control device in the peripheral intravenous catheter assembly 300 is illustrated, according to some embodiments. In some embodiments, the needleless syringe 150 may be inserted into the top of the injection port 145. In some embodiments, the needleless syringe 150 may be used for insertion of fluids into the peripheral intravenous catheter assembly 300 for administration of fluids to a patient. In some embodiments, the leaves 315 of the split septum 310 may be deformed downward and into the injection port 145 by the needleless syringe 150.

In some embodiments, the duckbill valve 320 may rest within the injection port 145. In some embodiments, the injection port 145 may have a structure that corresponds to the ring 330 of the duckbill valve 320. In some embodiments, the structure may hold the duckbill valve 320 in place within the injection port 145.

In some embodiments, a fluid may be injected into the peripheral intravenous catheter assembly 300 using the needleless syringe 150 inserted into the injection port 145. In some embodiments, the leaves 315 of the duckbill valve 320 may be opened by the force of the fluid. In some embodiments, the fluid may then flow into the lumen 155 of the catheter hub 110. In some embodiments, the fluid may then flow from the catheter hub 110 into the catheter 130 and into the patient.

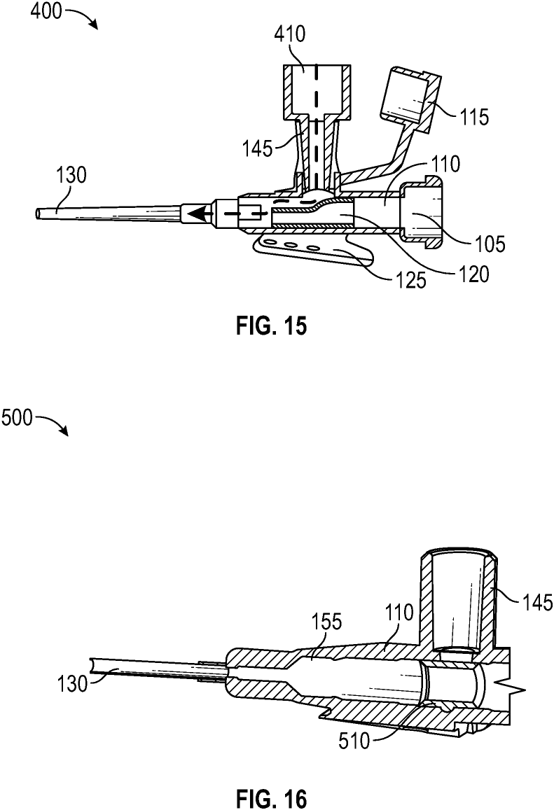

Referring now to FIG. 15, an injection port backflow control device in a peripheral intravenous catheter assembly 400 is illustrated, according to some embodiments. In some embodiments, the peripheral intravenous catheter assembly 400 may be similar or identical to one or more of the following in terms of one or more components and/or operation: the peripheral intravenous catheter assembly 100, the peripheral intravenous catheter assembly 200, and the peripheral intravenous catheter assembly 300. In some embodiments, flow of fluid into the peripheral intravenous catheter assembly 400 through the injection port 145 is illustrated by arrow 410, according to some embodiments. In some embodiments, a fluid may flow in through the injection port 145 and the force of the fluid may elastically deform a valve 120 such that the fluid may flow into the catheter 130 and into a patient. According to some embodiments, the valve 120 may deform downward when fluid is injected through the injection port 145 and then the valve 120 may return to its original cylindrical shape preventing fluids from flowing out of the injection port 145. In some embodiments, the peripheral intravenous catheter assembly 400 may include one or more of the following: an injection port cap 115, a catheter hub 110, an end cap 105, and wings 125.

In some embodiments, the valve 120 may slide in a peripheral direction away from the catheter 130 due to the force of fluids flowing into the catheter 130 from the patient and then from the catheter 130 into the catheter hub 110. In some embodiments, the displaced valve 120 may not cover the injection port 145, thus allowing fluids to leak from the catheter hub 110 out of the injection port 145. Various methods for preventing displacement of the valve 120 are described herein.

Referring now to FIG. 16, an injection port backflow control device in a peripheral intravenous catheter assembly 500 is illustrated, according to some embodiments. In some embodiments, the peripheral intravenous catheter assembly 500 may be similar or identical to one or more of the following in terms of one or more components and/or operation: the peripheral intravenous catheter assembly 100, the peripheral intravenous catheter assembly 200, the peripheral intravenous catheter assembly 300, and the peripheral intravenous catheter assembly 400. In some embodiments, the peripheral intravenous catheter assembly 500 may include one or more of the following: the catheter 130, the catheter hub 110, the lumen 155 within the catheter hub 110, the injection port 145, and a valve 510 with a protrusion. In some embodiments, the protrusion may correspond to an indent within the catheter hub 110. In some embodiments, the protrusion may prevent the valve 510 from being displaced within the catheter hub 110. In some embodiments, the valve 510 may prevent fluids from flowing up and out through the injection port 145. In some embodiments, the valve 510 may compress to allow fluids to flow from the injection port 145 into the catheter hub 110.

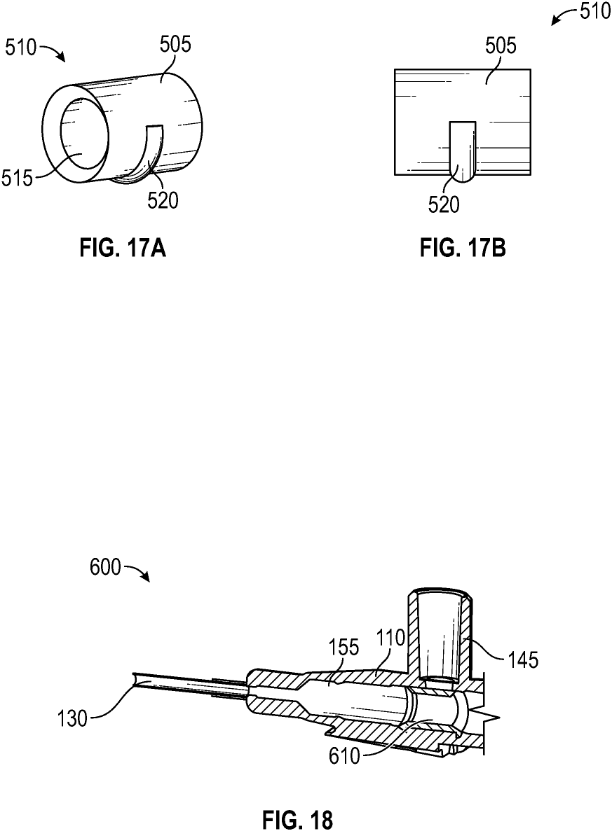

Referring now to FIGS. 16-17, a protrusion 520 is illustrated, according to some embodiments. In some embodiments, the protrusion 520 may be positioned on an outer surface 505 of the valve 510. In some embodiments, the protrusion 520 may be positioned in the center, off center, or on a side of the valve 510. In some embodiments, the protrusion 520 may partially encircle or fully encircle the valve 510. In some embodiments, the protrusion 520 may have any shape. In some embodiments, the protrusion 520 may include a semi-annular arc shape, which may facilitate insertion of the valve 510 into the catheter hub 110.

In some embodiments, the valve 510 may include an outer surface 505 that is slightly smaller or the same size as an inner diameter of the lumen 155. In some embodiments, the lumen 155 may have a corresponding indentation that couples with the protrusion 520. In some embodiments, the protrusion 520 coupling with the indentation may prevent displacement of the valve 510. In some embodiments, the valve 510 may be retained in its position within the lumen 155 by friction.

Referring now to FIG. 18, an injection port backflow control device in a peripheral intravenous catheter assembly 600 is illustrated, according to some embodiments. In some embodiments, the peripheral intravenous catheter assembly 600 may be similar or identical to one or more of the following in terms of one or more components and/or operation: the peripheral intravenous catheter assembly 100, the peripheral intravenous catheter assembly 200, the peripheral intravenous catheter assembly 300, the peripheral intravenous catheter assembly 400, and the peripheral intravenous catheter assembly 500. The peripheral intravenous catheter assembly 600 may include one or more of the following: the catheter 130, the catheter hub 110, the lumen 155 within the catheter hub 110, the injection port 145, and the valve 610 with multiple protrusions. The protrusions may correspond to one or more indents within the catheter hub 110. In some embodiments, the protrusions may prevent the valve 610 from being displaced within the catheter hub 110. In some embodiments, the valve 610 may prevent fluids from flowing up and out through the injection port 145. In some embodiments, the valve 610 may compress to allow fluids to flow from the injection port 145 into the catheter hub 110.

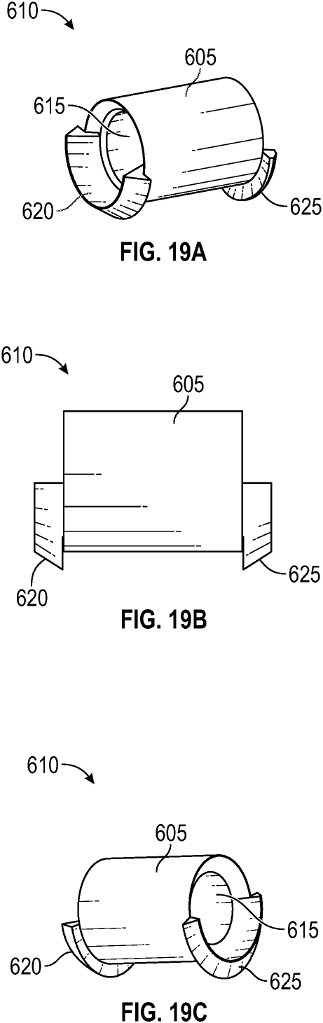

Referring now to FIGS. 18-19, the valve 610 with a first protrusion 621 and a second protrusion 625 is illustrated, according to some embodiments. In some embodiments, the first protrusion 621 may include or correspond to the protrusion 620. As illustrated in FIG. 19A, in some embodiments, the first protrusion 621 and second protrusion 635 may be positioned on an outer surface 605 of the valve 610. In some embodiments, the first protrusion 621 may be positioned on a first edge of the valve 610. In some embodiments, the second protrusion 625 may be positioned on a second edge of the valve 610. In some embodiments, the first protrusion 621 and second protrusion 625 may partially encircle or fully encircle the valve 610.

In some embodiments, the first protrusion 621 and second protrusion 625 may have any suitable shape. In some embodiments, the first protrusion 621 and the second protrusion 625 may include a semi-annular arc shape, which may facilitate insertion of the valve 510 into the catheter hub 110. In some embodiments, the valve 610 may include an outer surface 605 that is slightly smaller or the same size as an inner diameter of the lumen 155. In some embodiments, the lumen 155 may have one or more corresponding indentation that couples with the first protrusion 621 and/or the second protrusion 625. In some embodiments, the first protrusion 621 and/or the second protrusion 625 coupling with the indentation may prevent displacement of the valve. 510. In some embodiments, the valve 610 may be retained in its position within the lumen 155 by friction.

FIG. 19B is a side view of the valve 610, according to some embodiments. In some embodiments, the first protrusion 621 may have a slightly larger width than the second protrusion 625. In some embodiments, the first protrusion 621 and the second protrusion 625 may have the same width. In some embodiments, the second protrusion 625 may have a larger width than the first protrusion 621. FIG. 19C is another view of the valve 610 with the first protrusion 621 and the second protrusion 625, according to some embodiments.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A peripheral intravenous catheter assembly, comprising:
a catheter;
a catheter hub coupled to the catheter;
an injection port disposed on the catheter hub; and
an injection port backflow control device, comprising:
a concentric port;
a bellow; and
a duckbill valve.

2. The peripheral intravenous catheter assembly of claim 1, wherein the injection port is configured to receive a needleless syringe.

3. The peripheral intravenous catheter assembly of claim 1, wherein the injection port backflow control device is configured to permit fluids to flow into the catheter hub and prevent fluids from flowing out of the catheter hub through the injection port.

4. The peripheral intravenous catheter assembly of claim 1, wherein the bellow is vertically compressible.

5. The peripheral intravenous catheter assembly of claim 1, wherein the bellow blocks a sidewall opening of the concentric port when the bellow is in an expanded state.

6. The peripheral intravenous catheter assembly of claim 1, wherein the bellow is compressible to permit fluid to flow into a sidewall opening of the concentric port.

7. The peripheral intravenous catheter assembly of claim 1, wherein the bellow surrounds the concentric port and covers a sidewall opening in the concentric port when the bellow is not compressed, the concentric port comprising a vertical opening connecting the sidewall opening with an opening in the base of the concentric port, the opening in the base of the concentric port connecting with an opening in the duckbill valve, the duckbill valve opening to permit a fluid to flow into the catheter hub.

8. The peripheral intravenous catheter assembly of claim 1, wherein a fluid pathway within the injection port backflow control device is defined by a sidewall opening in the concentric port, an annular opening in the center of the concentric port connecting the sidewall opening to the top of the duckbill valve, the duckbill valve expanding to permit passage of a fluid into the catheter hub.

9. A peripheral intravenous catheter assembly, comprising:
a catheter;
a catheter hub coupled to the catheter;
an injection port disposed on the catheter hub; and
an injection port backflow control device, comprising:
a split septum; and
a duckbill valve.

10. The peripheral intravenous catheter assembly of claim 8, wherein the injection port is configured to receive a needleless syringe.

11. The peripheral intravenous catheter assembly of claim 9, wherein the injection port backflow control device is configured to permit fluids to flow into the catheter hub and prevent fluids from flowing out of the catheter hub through the injection port.

12. The peripheral intravenous catheter assembly of claim 9, wherein the duckbill valve is placed within the injection port, a line formed by a meeting of opposing lips of the duckbill valve oriented downward towards the catheter hub.

13. The peripheral intravenous catheter assembly of claim 9, wherein the split septum is placed as a cap over a top opening of the injection port, leaves of the split septum configured to be deformable to open downwards towards the catheter hub.

14. The peripheral intravenous catheter assembly of claim 9, wherein a fluid pathway through the injection port and into the catheter comprises, an opening in the split septum connected to an opening in the duckbill valve, the duckbill valve expanding to allow a fluid to flow into the catheter hub and through the catheter hub into the catheter.

15. A peripheral intravenous catheter assembly, comprising:
a catheter;
a catheter hub coupled to the catheter;
an injection port disposed on the catheter hub; and
an injection port backflow control device, comprising a deformable annular valve disposed around an inner surface of the catheter hub and blocking an opening in the catheter hub connected to the injection port.

16. The peripheral intravenous catheter assembly of claim 15, wherein the injection port is configured to receive a needleless syringe.

17. The peripheral intravenous catheter assembly of claim 15, wherein the injection port backflow control device is configured to permit fluids to flow into the catheter hub and prevent fluids from flowing out of the catheter hub through the injection port.

18. The peripheral intravenous catheter assembly of claim 15, wherein the annular valve is configured to deform longitudinal from a fluid pressure of a fluid injected through the injection port into the catheter hub.

19. The peripheral intravenous catheter assembly of claim 14, wherein the annular valve further comprises a projection from an outer sidewall of the annular valve.

20. The peripheral intravenous catheter assembly of claim 18, wherein the catheter hub comprises a notch that corresponds to the projection on the annular valve, the notch and the projection engaging to secure the annular valve within the catheter hub.
